(19) United States
(12) Patent Application Publication
(10) Pub. No.: US 2003/0118614 A1
(43) Pub. Date: Jun. 26, 2003

(54) Water-in-oil polymer dispersion as additive in active ingredient-comprising compositions

(76) Inventors: Ewald Sieverding, St. Johann (DE); Sandra Hintz, Krefeld (DE); Thomas Dambacher, Grefrath (DE); Thomas von Beckerath, Krefeld (DE); Johannes Busch, Meerbusch (DE)

Correspondence Address: FROMMER LAWRENCE & HAUG LLP, 745 Fifth Avenue, New York, NY, 10151, US

(21) Appl. No.: 10/216,131

(22) Filed: Aug. 9, 2002

(30) Foreign Application Priority Data
Aug. 13, 2001 (DE) 101 38 382.7

(51) Int. Cl. A61K 9/00

(52) U.S. Cl. 424/400

(57) ABSTRACT

The invention relates to a composition comprising

(A) an additive in an amount in a range of from 0.001 to 2% by weight, based on the total weight of the composition,

(B) at least one active ingredient which differs from the additive (A), and

(C) water in an amount of at least 50% by weight, based on the total weight of the composition,

to a process for the preparation of the composition according to the invention, to the composition obtainable by the process according to the invention, and to the use of the composition according to the invention in agriculture, in forestry, in horticulture, in fruit production, in the control of vectors, in plant growing, in plant breeding, in connection with seed, plant materials, nonagricultural applications, for controlling or combating organisms, and in connection with the storage or processing of fruits and crops or plant materials.

### RELATED APPLICATIONS

This application claims priority to German application No. 101 38 382.7, filed on Aug. 13, 2001, herein incorporated by reference.

### BACKGROUND OF THE INVENTION

1. Field of the Invention

The present invention relates to a composition, to a process for the preparation of a composition, to a composition obtainable by this method, to the use of a water-in-oil polymer dispersion, to a method of controlling an organism, to a method of regulating the growth of plants, and to the use of the composition.

2. Description of the Related Art

DE 199 36 223 A1 discloses an active ingredient-comprising composition comprising a water-in-oil polymer dispersion and at least one active ingredient. The active ingredients are added before, during or after polymerization. Owing to the active ingredient-comprising composition described in DE 199 36 223 A1, a reduced what is known as the run-off effect and a reduced what is known as the leaching effect are observed when the composition is applied to substrates. However, a markedly increased activity of the active ingredient owing to the compositions described in this document has not been found. A disadvantage of these compositions is that relatively large amounts of both active ingredient and water-in-oil polymer dispersion have to be employed.

### OBJECTS OF THE INVENTION

The invention is generally based on the object of overcoming the disadvantages of the prior art.

The object according to the invention furthermore consists in describing a composition with the aid of which the activity of active ingredients can be increased and the use concentration of these active ingredients can therefore be reduced.

A further object according to the invention consisted in improving the availability, to plants, of active ingredients.

### SUMMARY OF THE INVENTION

The objects stated above are achieved by a composition comprising

(A) an additive in an amount in a range of about 0.001 to about 2% by weight, preferably in a range of about 0.01 to about 1% by weight and very particularly preferably in a range of about 0.25 to about 0.75% by weight, in each case based on the total weight of the composition, and

(B) at least one active ingredient which differs from the additive (A), and

(C) water in an amount of at least about 50% by weight, preferably at least about 75% by weight, and very particularly preferably at least about 90% by weight based on the total weight of the composition,

the additive (A) being from a water-in-oil polymer dispersion comprising

(A1) a crosslinked polymer in an amount in a range of about 10 to about 70% by weight, preferably in a range of from about 20 to about 50% by weight and very particularly preferably in a range of from about 25 to about 35% by weight, in each case based on a total weight of the additive (A),

(A2) a hydrophobic organic fluid in an amount in a range of from about 20 to about 80% by weight, preferably in a range of from about 25 to about 50% by weight, and very particularly preferably in a range of from 30 to 40% by weight, in each case based on the total weight of the additive (A),

(A3) one or more water-in-oil emulsifiers in an amount in a range of from about 0.5 to about 10% by weight, preferably in a range of from about 1 to about 8% by weight, and very particularly preferably in a range of from about 2 to about 6% by weight, in each case based on the total weight of the additive (A),

(A4) one or more oil-in-water emulsifiers in an amount in a range of from about 0.5 to about 10% by weight, preferably in a range of from about 1 to about 8% by weight, and very particularly preferably in a range of from about 2 to about 6% by weight, in each case based on the total weight of the additive (A),

(A5) one or more additives in an amount in a range of from 0 to about 20% by weight, preferably in a range of from about 0.01 to about 10% by weight, based on the total weight of the additive (A), and

(A6) water in such an amount that the total of (A1) to (A6) is 100% by weight.

### DETAILED DESCRIPTION OF THE INVENTION

In a preferred embodiment of the composition according to the invention, the additive (A) in a composition consisting of water and the additive in an amount of 0.5% by weight based on the total weight of the composition have at least one of the following characteristics described in accordance with the test methods described herein:

(a1) reduced flow rate of the composition by at least 10%, preferably by at least 25%, in each case in comparison with the flow rate of water;

(a2) extended period of time which elapses until the water present in 10 g of the composition has evaporated at 22° C. and a pressure of 1 bar in comparison with the period of time which elapses until the same amount of water without the additive has evaporated under identical conditions by at least 1 hour.

In a further preferred embodiment of the composition according to the invention, the additive (A) in a composition consisting of water and the additive in an amount of 1.5% by weight based on the total weight of the composition have at least one of the following characteristics described in accordance with the test methods described herein:

(a1) reduced flow rate of the aqueous mixture by at least 75%, preferably by at least 95%, in each case in comparison with the flow rate of water;

(a2) extended period of time which elapses until the water present in 10 g of the composition has evaporated at 22° C. and a pressure of 1 bar in comparison with the period of time which elapses until the same amount of water without the additive has evaporated under identical conditions by at least 2 hours.

Suitable active ingredients (B) which differ from the additive (A) are preferably acaricides (AC), algaecides (AL), attractants (AT), repellents (RE), bactericides (BA), fungicides (FU), herbicides (HB), insecticides (IN), molluscicides (MO), nematicides (NE), rodenticides (RO), safeners (SA), sterilants (ST), synergists (SY), viricides (VI), growth regulators (PG) or at least two of these. Among these, HB, FU, IN, NE or PG are preferred, and FU, IN or PG are particularly preferred.

In accordance with the preferred embodiment, the term active ingredients are understood as meaning all of the active ingredients which are known to the skilled worker which are suitable, with the exception of tebuconazole, imidacloprid, thiacloprid, trifloxystrobin and iprovalicarb.

For the purposes of the present invention, growth regulators (PG) are substances which reduce the growth of a plant (without the fruit-bearing part) when brought into contact with the growth regulator (PG) compared with the growth of the same plant when not brought into contact with the growth regulator (PG).

In a preferred embodiment of the invention, the composition comprises at least two, preferably different, active ingredients (B) which differ from the additive (A).

In another preferred embodiment of the invention, the composition comprises at least three, preferably different, active ingredients (B) which differ from the additive (A).

In another preferred embodiment of the invention, the composition comprises at least four, preferably different, active ingredients (B) which differ from the additive (A).

Some preferred classes of active ingredients, preferred active compounds or preferred organisms as active ingredients (B) which differ from the additive (A) together with their use(s) are stated hereinbelow:

abamectin AC, IN; AC 375839 FU; AC 92553, HB; acephat IN; AC 382042 FU; acequinocyl AC; acetamiprid IN; acetochlor HB; N-acetylthiazolidine-4-carboxylic acids; acetoprol AC; acilflorfen-Na HB; acibenzolar-S-methyl FU; acifluorfen HB; aclonifen HB; acridine-bases RE; acrinathrin IN, AC; alachlor HB; alanycarb IN; aldicarb AC, NE, IN; aldimorph FU; alkyldimethylbenzylammonium chloride HB; alkyldimethylethylbenzylammonium chloride HB; alkyltrimethylammonium chloride BA FU; alkyltrimethylbenzylammonium chloride HB; allidochlor, HB; alloxydim HB; allyl alcohol HB; alphacypermethrin (=α-cypermethrin); alphamethrin (=α-cypermethrin) IN: aluminum ammonium sulfate RE; aluminum phosphide IN, RO; aluminum sulfate MO, PG; ametryn HB; amicarbazon HB; amidosulfuron HB; amino acids PG; 2-aminobutane FU; amitraz AC, IN; amitrole (=aminotriazole) HB; ammonium carbonate FU; ammonium hydroxide FU; ammonium sulfamate HB; ammonium sulfate HB; ammonium thiocyanate HB; amosulfuron HB; ampropylfos FU; ancymidol PG; andoprim FU; anilazine FU; anilofos HB; anthracene oil IN, AC, HB, RO; anthraquinone RE; arsenic anhydride; asulam HB; atrazine HB; azaconazole IN, FU; azadirachtin IN; azafenidin FU, HB; azamethiphos IN; azinphos-methyl IN, AC; aziprotryn HB; azocyclotin AC; azoxystrobin FU; BAS 500F; barban HB; barium fluorosilicate IN; barium nitrate RE; barium polysulfide IN, FU; BAY MKH 6561 HB; beflubutamid HB; benfluamid HB; benalaxyl FU; benazolin (-ethyl) HB; bendiocarb IN; benefin HB; benbiclon/benzobicyclon HB; benfluralin HB; benfuracarb IN, NE; benfuresate HB; benodanil FU; benomyl FU; bensulfuron (-methyl) HB; bensulid HB; bensultap IN; bentaluron FU; bentazone HB; benzalconium chloride HB; benzamacril FU; benzoximate AC; benzoylprop (-ethyl) HB; benzthiazuron HB; 6-benzyladenine PG; benzofenap HB; benzofluor PG, HB; 2-benzyl-4-chlorophenol FU; bialaphos HB, FU; bifenazate AC, IN; bifenox HB; bifenthrin IN, AC; biloxazol FU; binapacryl AC, FU; bioallethrin IN; bioresmethrin IN; biphenyl FU; bistrifluron IN; bitertanol 386 FU; blasticidin-S FU; Bordeaux mixture=calcium copper sulfates FU; boric acid IN; BPMC IN; brandol FU; brassinolide PG; brodifacoum RO; brofenprox=halfenprox; bromacil HB; bromadiolone RO, RE; bromethalin RO, RE; bromobutid HB; bromocyclen AC, IN; bromofenoxim HB; bromophos IN; bromophos-ethyl IN; bromopropylate AC; bromoxynil HB; bromuconazole FU; bronopol FU, BA; buminafos PG, HB; bupirimate FU; buprofezin AC, IN; Burgundy mixture FU; butachlor HB; butam (=tebutam); butenachlor HB, FU; buthidazole HB; buthiobate FU; butocarboxim IN; butoxycarboxim IN, AC; butralin HB, PG; buturon HB; sec-butylamine FU, IN; cadusafos IN, NE; calciferol RO; calcium carbide RE; calcium cyanamide HB; calcium chloride FU, PG; calcium copper oxychloride FU; calcium copper sulfate FU; calcium cyanide (see cyanides) IN, RO; calcium phosphate RO; calcium phosphide RO; caloxidim HB; captan FU; captafol FU; carbaryl IN, PG; carbatene (see metiram); carbendazim FU; carbetamide HB; carbofuran IN, NE, AC; carbon dioxide IN, RO; carbon disulfide IN, NE; carbophenothion IN, AC; carbosulfan IN, NE; carboxin FU; cartap (hydrochloride) IN; carpropamid FU; carvon FU; cetrimid HB; quinomethionate AC, FU; chlomethoxyfen HB; chloral- semi-acylal HB; chloralose RO; chloramben HB; chlorates HB; chlorbenzthiazon FU; chlorbromuron HB; chlorbufam HB; chloretazate PG; chlorethoxyfos IN; chlorfenapyr AC, IN; chlorfenazol FU; chlorfenprop (-methyl) HB; chlorfenson IN, AC; chlorfena HB; chlorfenvinphos AC, IN; chlorfluazuron IN; chlorflurenol (chlorflurecol) HB, PG; chloridazon HB; chlormephos IN; chlormequat (-chloride) PG; chlorobenzilate AC; chlorobufam HB; chlorofenizon=chlorfenson; 4-chloro-3-methylphenol FU; chloroneb FU; p-chloronitrobenzene IN; chlorophacinon RO; chlorophenprop-methyl HB; chlorophyllin FU, BA; chloropicrin NE, IN, FU; chloropropylate AC; chlorothalonil FU; chloroxuron HB; chlorphonium chloride PG; chlorpropham PG, HB; chlorpyrifos IN, AC; chlorpyrifos-methyl IN, AC; chlorsulfuron HB; chlorthaldimethyl HB; chlorthiamid HB; chlorthiamidin IN; chlorthiophos IN; chlortoluron HB; chlozolinate FU; cholecalciferol RO; cholin chloride RO; chromafenozide IN; cimetacarb-ethyl (see trinexapac); cimidon-ethyl HB; cinmethylin HB; cinidon-ethyl HB; cinosulfuron HB; cintofen PG; citronellol IN, RE; cizzeathin PG; clefoxydim HB; clethodim 508 HB; clodinafop (-propargyl) HB; clofencet PG; clofentezine AC; clomazone HB; clomeprop HB; clopoxydim HB; clopyralid HB; clozylacon FU; codlemone (cf. 8,10-dodecadien-1-ol) AT; copper acetate FU; copper ammonium carbonate FU; copper β-cyclodextrin hydroxide FU; basic copper carbonate FU; copper hydroxide BA, FU; copper naphthenate FU, RE; copper oxychloride FU; copper salts of the fatty and rosinic acids FU; copper sulfate AL, FU; tribasic copper sulfate AL, FU; coumachlor RO; coumafuryl RO; coumaphen (cf. warfarin); coumatetralyl RO; 4-CPA (4-chlorophenoxyacetic acid) PG; p-cresyl acetate RE; cresylic acid ST, FU; crimidine RO; cubiet FU; cufraneb FU; cuprammonium FU; cuprous oxide FU; cyanamide PG, HB; cyanazine HB; cyanide IN, RO; cycloate HB; cycloxydim HB; cycloheximide FU; cycloprothrin IN; cycluron HB; cyflufenamid FU; cyfluthrin IN, AC; β-cyfluthrin IN; λ-cyhalothrin IN; cyhexatin AC; cymoxanil FU; cyprazine HB; cypermethrin IN, AC; α-cypermethrin IN; ζ-cypermethrin IN; cyproconazole FU; cyprodinil FU; cyprofuram FU; cyromazine IN; cystein PG; 2,4-D HB PG; DADZ (zinc dimethyldithiocarbamate) RE; dalapon (-sodium) HB; daminocide PG; Daphne oil RE; dazomet NE, FU, HB, IN, ST; 2,4-DB HB; DCPA HB; D-D NE, FU, IN; debacarb FU; 1-decanol PG; 5-decen-1-ol AT; 5-decen-1-yl acetate AT; decanoic acid; DEH 112 FU, HB; deltamethrin IN; demeton-S-methyl IN, AC; demeton-S-methylsulfone IN; denathonium benzoate RE; desmedipham HB; desmetryn HB; diafenthiuron IN, AC; dialifos IN, AC; di-allate HB; diammonium phosphate AC; diazinon IN, AC; dicamba HB; dichlobenil HB; dichlofenthion NE, IN; dichlofluanid FU; dichlone FU; dichloran FU; p-dichlorobenzene RO; methyl dichlorobenzoate FU; dichlorophen AL, BA, HB, FU; 1,2-dichloropropane IN, FU, ST; 1,3-dichloropropene NE, HB; 1,3-(cis-)dichloropropene NE, HB; dichlorprop HB; dichlorprop-P HB; dichlorvos IN, AC; diclobutrazol FU; diclofop(-methyl) HB; diclocymet FU; dicloran FU; diclomezine FU; dicofol AC; dicrotophos IN, AC; dicyclopentadiene RE, PG; didecyl dimethylammonium chloride FU; dienochlor AC, diethatyl (-ethyl) HB; diethion (see ethion); diethofencarb FU; difenacoum RO; difenamid=diphenamid; difenoconazole FU; difenoxuron HB; difenzoquat (-methylsulfate) HB, FU; difethialon RO; diflubenzuron IN; diflufenican HB; diflufenzopyr HB; diflumetorim FU; dimefluazole FU; dikegulac (-sodium) PG; dimefox AC, IN; dimefuron HB; dimepiperate HB; di-methyl-p-menthene PG; dimethachlor HB; dimethenamide HB; dimethipin PG, HB; dimethirimol FU; dimethoate IN, AC; dimethomorph FU; dimeturon HB; 3,7-dimethyl-2,6-octadienal AT; 3,7-dimethyl-2,6-octadien-1-ol AT; dimexano HB; dimoxystrobin FU; dinicona-zole FU; dinitramine HB; dinobuton AC, FU; dinocap FU, AC; dinosep acetate HB; dinoterb HB; dioctyl dimethylammonium chloride FU, BA; dioxacarb IN; 1,7-dioxaspiro-5,5-undecane AT; dioxathion AT; diphacinone RO; diphenamid HB; DNOC IN, AC, HB; diphenylamine FU, PG; 1,3-diphenylurea PG; diquat (dibromide) HB; (disodium) ethylenediaminetetraacetate and its salts HB; dipyrithion BA, FU; disodium octaborate HB; disulfoton AC, IN; ditalimfos FU; dithianon FU; 2-(dithiocyanomethylthio)benzothiazole FU; diuron HB; DNOC IN, AC, FU, HB; (4Z,9Z)-7,9-dodecadien-1-ol AT; (E)7-(Z)9-dodecadienyl acetate AT; (Z)-8-dodecanol AT; (Z)-5-dodccenyl-1-yl acetate AT; (Z)-8-dodecenyl acetate AT; (E/Z)-8-dodecenyl acetate AT; (Z)-9-dodecenyl acetate 422 AT; (E)-10-dodecenyl acetate AT; trans-9-dodecyl acetate AT; dodecyl alcohol AT; dodemorph FU; dodine FU; DPX IN, HB; drazoxolon FU; dymron HB; ebufos (cf. cadusafos); eglinazine-ethyl HB; edifenphos FU; emamectin benzoate IN; endosulfan IN, AC; endothal AL, PG, HB; Bacillus thuringiensis δ-endotoxin IN; enilconazole (cf. imazalil); epocholeon PG; epoxiconazole FU; 7,8-epoxi-2-methyl-octadecane AT; EPTC HB; esfenvalerate IN; esprocarb HB; etacelasil PG; etaconazole FU; ethaboxam FU; ethalfluralin HB; ethanethiol RO; ethanol MO; ethephon PG; ethidimuron HB; ethiozin HB; ethiofencarb IN; ethion IN, AC; ethoxyfen HB; ethirimol FU; ethoate-methyl AC, IN; ethofumesate HB; ethoprophos NE, IN; ethoxyquin FU, PG; ethyl hexanoate FU, BA; ethyl oleate PG; etofenprox IN; etoxazole AC, IN; etridiazole FU; etrimfos IN, AC; eucalyptus oil RE; famesol AT, famoxadon FU; fatty acid esters IN, PG; sodium salts of the fatty acids HB, FU, IN; fatty alcohols PG; fenapanil FU; fenamidon FU; fenaminosulf FU; fenamiphos NE; fenarimol FU; fenazaflor AC; fenazaquin AC; fenbuconazole FU; fenbutatin oxide AC; fenfuram FU; fenhexamid FU; fenitropan FU; fenitrothion IN, AC; fenizon=fenson; fenoprop PG, HB; fenothiocarb IN, AC; fenoxanil FU; fenoxaprop (-ethyl) HB; fenoxaprop-P (-ethyl) HB; fenoxycarb IN; fenpiclonil FU; fenpropathrin IN, AC; fenthiapropyl-etyl HB; fenpropidin FU; fenpropimorph FU; fenpyroximate AC; fenridazon PG; fenson AC; fenthion IN; fenthiosulf IN; fentin AL, MO, FU; fentin acetate FU, AL, MO, HB; fentin hydroxide FU, AL, MO, HB; fenuron HB; fenvalerate IN, AC; ferbam FU; ferimzone FU; fipronil IN; flamprop (-isopropyl) HB; flamprop (-methyl) HB; flamprop-M (-isopropyl) HB; flazasulforon HB; flocoumafen RO; flonicamid IN; fluacrypyrim AC; fluazifop (-butyl) HB; fluazifop-P (-butyl) HB; fluazinam FU; flubenzimine AC; flucarbazone HB; fluchloralin HB; flucycloxuron IN, AC; flucythrinate IN; fludioxonyl FU; flufenoxuron AC, IN; flumequine BA; flumetover FU; flufenacet HB; flufenpyr HB; flumethralin PG; fluometuron HB; flumioxazin HB; flumipropyn HB; flumicloralyl HB; flumetsulam HB; fluorbentranil HB; fluoroacetamid RO; fluorodifen HB; fluoroglycofen (-ethyl) HB; flupoxam HB; fluproxyfen IN; fluquinconazole FU; flurenol (flurecol) HB; fluridon HB; fluorochloridon HB; fluoroimid FU; fluroxypyr HB; flurprimidol PG; flurtramon HB; flusilazole FU; flusulfamid FU; flutenzin IN; flutolanil FU; flutriafol FU; (tau)fluvalinate AC, IN; focus HB; folic acid PG; folpet FU; fomesafen HB; fonofos IN; foramsulfuron HB; formaldehyde FU, BA, ST; formetanate IN, AC; formic acid IN; formothion IN, AC; fosamine (-ammonium) HB; fosetyl (-aluminum) FU; fosthiazate NE, IN; fosthietan IN, NE; formamsulfuron HB; fuberidazole FU; furalaxyl FU; furametpyr FU; furacarbonil FU; furathiocarb IN; cis-furconazole FU; furathiocarb IN; furconazole FU; furfural HB; furmecyclox FU; furyloxyfen HB; gelantine IN; gentian violet BA; gibberellic acid PG; gibberellin PG; glufosinate (-ammonium) HB; glutaraldehyde FU, BA; glutardialdehyde (cf. glutaraldehyde); glyphosate (including trimesium salt) HB; glyphosate salts HB (trimesium) (cf. glyphosate); guazatine FU, RE; halfenprox AC; halofenozide IN; haloxyfop HB; haloxyfop-R HB; haloxyfop-P-methyl HB; haloxyfop-ethoxyethyl HB; halosulfon HB; heptenophos IN; HW 52 HB; hexachlorobenzene FU; hexachlorophen FU; hexaconazole FU; cis-7,trans-11-hexadecadienyl acetate AT; (Z)-11-hexadecanol AT; Z-9-hexadecenal AT; (7Z-11Z)-7,11-hexadien-1-yl acetate AT; hexaflumuron IN; hexazinon HB; hexythiazox AC, IN; hydramethylnon IN; hydrolyzed proteins AT; hydroxyisoxazole FU; hydroxy-MCPA PG; hydroxyphenylsalicylamide FU; 8-hydroxyquinoline sulfate BA, FU; hymexazol FU; IKF-916 FU; ILIA 0051 HB; imazalil FU; imazamethabenz (-methyl) HB; imazapyr HB; imazaquin HB, PG; imazethabenz HB; imazethapyr HB, PG; imibenconazole FU; imidacloprid IN; iminoctadine FU; indolebutyric acid PG; indolyl acetic acid PG; indoxacarb IN; iodocarb FU; iodofenphos AC, IN; iodosulfuron HB; ioxynil HB; ipconazole FU; iprobenfos FU; iprovalicarb FU; iprodione FU; iron disodium EDTA HB; iron(II) sulfate HB, MO; iron(III) sulfate HB; isazofos IN, NE, HB; isocarbamid HB; isofenphos IN; isolan IN; isoprocarb IN; isopropalin HB; isoprothiolan FU; isoproturon HB; isouron HB; isoval RO; isovaledione FU; isoxaben HB; isoxathion IN; isoxapyrifop HB; JC 940 HB; karbutilate HB; kasugamycin FU, BA; KH 218 HB; kinoprene IN; kitazin P FU; kresoxim-methyl FU; KZ-165 FU; lactic acid PG; lactofen HB; lambda-cyhalothrin IN; lauryl dimethylbenzylammonium bromide FU, BA; lauryl dimethylbenzylammonium chloride HB; lecithin FU; lenacil 163 HB; lime phosphate PG; lime sulfur 17 FU, IN, AC; lindan IN, RO; linuron HB; lufenuron AC, IN; magnesium phosphide IN, RO; malathion IN, AC; maleic hydrazide PG; mancopper (“man” for Mn) FU; mancozeb FU; maneb FU; MCPA HB; MCPA-thioethyl HB, MO; MCPB HB; mecarbam IN, AC; mecoprop HB; mecoprop-P HB; MCPA HB; MCPB HB; mefenacet HB; mefenoxam FU; meferimzone FU; mefluidid PG, HB; mepanipyrim FU; mefluidide HB, PG; mephospholan IN; mepronil FU; merphos PG; mesosulfuron HB; metalaxyl FU; metalaxyl-M FU; metaldehyde MO; metam (-sodium) FU, IN, HB, NE; metamitron HB; metazachlor HB; metconazole FU; methabenzthiazuron HB; methacrifos AC, IN; methamidophos IN, AC; methasulfocarb PG, FU; methazole HB; methfuroxam FU; methidathion IN, AC; methiocarb IN, AC, MO, RE; methomyl AC, IN; methoprene IN; methoprothryne HB; methoxychlor IN; methoxyfenozid IN; methylarconic acid (cf. MSMA); methyl bromide FU, IN, NE, HB; methylene bisthiocyanate FU; 7-methyl-3-methylene-7-octen-1-yl AT; methylnaphthylacetamide PG; methylnaphthylacetic acid PG; methyl trans-6-nonenoate AT; methyl nonyl ketone RE; methyl isothiocyanate FU, NE, HB, IN; metiram FU; metobromuron HB; metolachlor HB; metomeclam FU; metominostrobin FU; metometuron (see tribenuron); metosulam HB; metoxuron HB; metrafenone FU, metribuzin HB; metsulfovax FU; metsulfuron (-methyl) HB; mevinphos IN, AC; milbemectin AC, IN; molinate HB; monalide HB; monocarbamide dihydrogen sulfate PG, HB; monocrotophos AC, IN; monolinuron HB; monuron HB; MSMA HB; MIT-466 IN; myclobutanil FU; myclozolin FU; nabam FU, HB, AL; naled IN, AC; naphthalene RE; 1-naphthylacetamide PG; 1-naphthylacetic acid PG; ethyl naphthyl acetate PG; naphthylacetic hydrazide PG; 2-naphtyloxy acetamide PG, HB; 2-naphthyloxyacetic acid PG; napropamid HB; naptalam HB; sodium thiocyanate (cf. sodium thiocyanate) neburon HB; neoasozin FU; (cf. 3,7-dimethyl-2,6-octadien-1-ol); nickel dimethyldithiocarbamate BA, FU; nicosulfuron HB; nicotin IN; nipyraclofen HB; nitenpyram IN; nitralin HB; nitrofen HB; nitrofuorfen HB; nitrothal (-isopropyl) FU; NNF-9850 FU; trans-6-nonen-1-ol AT; nonylphenol ether polyoxyethylene glycol PG; nonylphenol ethoxylate FU; norflurazon HB; noruron HB; novaluron IN; NSK 850 HB; nuarimol FU; (Z,Z)-octadienyl acetate AT; (Z)-13-octadecanol AT; octhilinone BA, FU; octyldecyldimethylammonium chloride FU, BA; ofurace FU; omethoate IN, AC; orbencarb HB; organomercury compounds FU; oryzalin HB; oxadiazon HB; oxadixyl FU; oxamocarb FU; oxasulfuron HB, FU, oxamyl AC, IN, NE; oxine-copper FU; oxycarboxin FU; oxydemeton-methyl IN, AC; oxyfluorfen HB; oxyquinolein FU; oxytetracyclin BA; paclobutrazole PG; papain RO; liquid paraffin HB, IN, AC; paraformaldehyde IN; paraquat HB; parathion IN, AC; parathion-methyl IN, RE; PCPA (cf. 4-CPA)pebulate HB; pefurazoate FU; pelargonic acid HB, PG; penconazole FU; pencycuron FU; pendimethalin HB; penoxsulam HB; pentachlorophenol IN, FU, HB; pentanochlor HB; 4-t-pentylphenol BA; pepper IN; perfluidon HB; permethrin IN; petroleum oils FU, HB, IN, AC; phenazine oxide FU; phenamiphos (cf. fenamiphos); phenisopham HB; phenmedipham HB; phenols HB, ST; phenothiol (see MCPA-thioethyl); phenothrin IN; phenthoate AC, IN; 2-phenylphenol (including the sodium salts) FU; pherodim AT; phorate IN; phosalone IN, AC; phosametine HB; phosdiphen FU; phosmet AC, IN; phosphamidon IN, AC; phosphoric acid phoxim IN; phthalide FU; picloram HB; picoxystrobin FU; picolinafen HB; piperophos HB; piperalin FU; pimaricin FU; pirimicarb IN; pirimiphos-ethyl IN; pirimiphos-methyl AC, IN; primisulforon HB; vegetable oils IN, AC, OT, FU, PG; polybutene IN; polyoxin FU; polyram FU; pyridalyl IN; potassium chlorate (cf. chlorates); potassium permanganate FU, BA, MO; potassium soap (cf. potassium salts of fatty acid); potassium sorbate FU; pretilachlor HB; primisulfuron HB; probenazole BA, FU; prochloraz FU; procyazin HB; procymidon FU; prodiamin HB; profenofos AC, IN; profluazole HB; profluralin HB; promecarb IN; prometon HB; prometryn HB; pronamide HB; pronumon AT; propachlor HB; propamocarb (-hydrochloride) FU; propanil HB; propham PG, HB; propaphos IN; propaquizafop HB; propargite AC, AT; propazine HB; propetamphos AC, IN; propham HB, PG; propiconazole FU; propineb FU; propionic acid FU, BA; propoxur IN; propyl 3-t-butylphenoxy acetate PG; propyzamide HB; pyrazolat HB; pyrazosulfuron-ethyl HB; prosulfocarb HB; prothiocarb FU; prothiofos IN; prothoate IN, AC; pymetrozine IN; pyraclofos IN, AC; pyraclonil HB; pyraflufen-ethyl HB; pyranocumarin RO; pyrazon (cf. chloridazon); pyrazolynate HB; pyrazophos FU; pyrazoxyfen HB; pyracarbolid FU; pyrethrins AC, IN; pyributicarb FU, HB; pyridaben AC, IN; pyridafenthion IN, AC; pyrifthalide HB; pyridate HB; pyrifenox FU; pyrimethanil FU; pyrimidifen AC, IN; pyriproxyfen IN; pyrnachlor HB; pyroquilone FU; pyroxyfur FU; quinconazole FU; quassia IN, RE; quinalphos AC, IN; quinclorac HB; quinmeral HB; quinoclamin HB, AL; quinoxyfen FU; quintozene FU; quinmerac HB; quinomethionate; quintozene FU; quizalofop HB; quizalofop-P HB; rabenzazole FU; rapeseed oil (cf. vegetable oils); renriduron (cf. rimsulfuron); resmethrin IN; RH-7281 FU; rimilure-8L (cf Z,E)-11-tetradecadien-1-yl acetate); rimsulfiron HB; rotenon AC, IN; scillirosid RO; marine algae extract PG; sebacic acid RE; secbumeton HB; seconal OT; serricornin AT; sethoxydim HB; siduron HB; silafluofen IN; silthiofam FU; silver iodide RE; silver nitrate PG, FU; simazine HB; simeconazole FU; sodium p-t-amyl phenate FU, BA; sodium p-t-amyl phenoxide FU; sodium arsenite FU, IN; sodium o-benzyl-p-chlorophenoxide FU; sodium chlorate (cf. chlorates); sodium chloride HB; sodium cyanide (cf. cyanides); sodium diacetone ketogulonate PG; sodium dichlorophenate FU, BA; sodium dimethyl arsinate HB, RO; sodium dimethyl dithiocarbamate FU; sodium dioctyl sulfosuccinate AC; sodium fluorosilicate IN; sodium hydrogen carbonate FU; sodium hydroxide HB; sodium hypochlorite BA; sodium lauryl sulfate FU, BA; sodium metabisulfite FU; sodium monochloroacetate HB; sodium o-nitrophenolate PG; sodium bis-p-nitrophenolate PG; sodium 5-nitroguaiacolate PG; sodium pentaborate PG; sodium 2-phenylphenate (cf. 2-phenylphenol); sodium propionate FU; sodium silver thiosulfate HB; sodium tetraborate IN, HB, MO; sodium tetrathiocarbamate NE; sodium tetrathiocarbonate NE, FU; sodium thiocyanate HB; sodium p-toluenesulfochloramide BA; epoxylated soya oil IN; silthiofam FU; spinosad IN; spinosyn IN; spirodiclofen AC; spiroxamin FU; SSF-126 FU; SSF-129 FU; streptomycin BA; strychnin RO; sulcotrion HB; sulfentranzon HB; sulfodiazol (cf. ethidimuron); sulfosate (cf glyphosate trimesium); sulfotep IN, AC; sulfur FU, AC, RE; sulfuric acid HB; sulprofos IN; 2,4,5-T HB; Tamagrowth (TS303) PG; tar acid IN; tar oils FU, IN, HB; 2,3,6-TBA HB; TCA HB; TCMTB FU; tebuconazole FU; tebufenozide IN; tebufenpyrad AC; tebupyrimiphos IN; tebutam HB; tebuthiuron HB; tecloftalam BA, FU; tecnazene FU, PG; teflubenzuron IN; tefluthrin IN; temephos IN; tepraloxydim HB; terbacil HB; terbucarb HB; terbuchlor HB; terbufos NE, IN; terbumeton HB; terbuthylazine HB; terbutryn HB; Terrazole FU; tefcyclasis FU; tetrachlorvinphos AC, IN; tetraconazole FU; (Z,E)-11-tetradecadien-1-yl acetate AT; (Z)-7-tetradecanol AT; (Z)-7-tetradecenal AT; (Z)-9-tetradecenyl acetate AT; (E)-11-tetradecenyl acetate AT; tetradifon AC, IN; tetramethrin IN; tetrasul AC; TH 913 HB; thallium sulfate RO; thiabendazole FU; thiacloprid IN; thiamethoxam IN; thiameturon see thifensulfuron, thiazafluron HB; thiazopyr HB; thidiazuron PG; thifensulfuron (-methyl) HB; thifluzamide FU; thiobencarb HB; thiocarb IN; thiocyclam (hydrogen oxalate) IN; thicyofen FU; thiodicarb MO, IN; thiofanox AC, IN; thiometon IN, AC; thionazin NE; thiophanate-ethyl FU; thiophanate-methyl FU; thiourea RO; thiram FU; tiadinil FU; tioxymid FU; tiocarbazil HB; tolclofos-methyl FU; tolylfluanid FU, AC; tolylphtalam PG; tralkoxydim HB; tralomethrin IN; transfluthrin IN; triadimefon FU; triadimenol FU; triapenthenol PG; triasulfuron HB; triazamate IN; triazbutil FU; triazofenamid HB; triazophos NE, IN, AC; triazoxide FU; tribenuron (-methyl) HB; S,S,S-tributyl phosphorotrithioate PG; tributyl phosphorotrithioite (cf. merphos); tributyltin oxide FU; tricalcium phosphate RO; trichlamide FU; trichlorfon IN; trichloronate IN; triclopyr HB; tricosene IN; tricyclazole FU; (4E,7Z)-4,7-tridecadien-1-yl acetate AT; tridemoprh FU; tridiphane HB; trietazine HB; trifenmorph MO; trifloxystrobin FU; trifloxysulfuron HB; triflumizol FU; triflumuron IN; trifluralin HB; triflusulfuron (-methyl) HB; triforine FU, AC; trimedlur AT; trimeturon HB; trinexapac PG; trioxymethylene FU; tritazin HB; triticonazole FU; urea FU; uniconazole PG, FU; validamycin FU; vamidothion IN, AC; Vapam IN, NE, FU; vemolate HB; vinclozolin FU; warfarin RO; wax RO; XRD-563 FU; xylyl methylcarbamate IN; xylachlor HB; xylylcarb IN; zarilamid FU; zetacypermethrin (cf. zeta-cypermethrin) IN; zinc phosphide IN, RO; zineb FU; zineb ethylenethiuram disulfide adduct (cf. (Z)-9-dodecenyl acetate); (Z)-11-tetradecen-1-yl acetate AT; bitumen pruning c, bone oil RE; lubricating grease IN; quartz sand RE; odor repellents RE; AD-67 safener SA; benoxacor safener SA; cloquintocet-mexyl safener SA; N,N-diallyl-2,2-dichloroacetamide (R 25 788) safener SA; extender syn. SY; fenchlorazole (-ethyl) safener SA; fenchlorim safener SA; flurazole safener SA; mefenpyr-diethyl safener SA; piperonyl butoxide syn. SY; sulfaquinoxalin syn SY.

In accordance with a further embodiment, biological crop protectants and pesticides are furthermore suitable as active ingredient (B). These preferably include bacteria, slime molds, nematodes, viruses or substances or other constituents thereof. Preferably suitable are Bacillus spp. (for example B. sphaericus IN, B. subtilis FU; B. thuringiensis IN with B. thuringiensis aizawai, B. thuringiensis israelensis, B. thuringiensis kurstaki, B. thuringiensis tenebrionis); pseudomonas spp., streptomyces griseoviridis FU; granulosis viruses IN or, for example, nuclear polyhedrosis viruses IN.

Embodiments of the composition which are preferred in accordance with the invention are those which comprise at least one of the abovementioned active ingredients, but not tebuconazole, imidacloprid, thiacloprid, trifloxystrobin and iprovalicarb. At least one of the active ingredients tebuconazole, imidacloprid, thiacloprid, trifloxystrobin and iprovalicarb, preferably all of the abovementioned active ingredients, are present in another embodiment, of the composition, which is preferred in accordance with the invention.

Preferably, the active ingredient (B) which differs from the additive (A) is present in the composition according to the invention in an amount in a range of from about 0.0001 to about 10% by weight, preferably in an amount in a range of from about 0.001 to about 5% by weight, in each case based on the total weight of the composition.

The crosslinked polymers (A1) which are present in the water-in-oil polymer dispersions according to the invention are a class of polymers which are preferably prepared by inverse emulsion polymerization, where finely distributed crosslinked polymers are generated in a continuous organic phase which is not miscible with water with addition of water-in-oil emulsifiers.

The crosslinked polymers (A1) are preferably water-soluble polymers.

As components, the polymer (A1) is preferably based on (α1) about 0.1 to about 99.999% by weight, preferably about 20 to about 98.99% by weight, and particularly preferably about 30 to about 98.95% by weight of polymerized, ethylenically unsaturated, acid-group-containing monomers or salts thereof or polymerized, ethylenically unsaturated monomers comprising a protinated or quaternized nitrogen, or mixtures of these,

(α2) 0 to about 70% by weight, preferably about 1 to about 60% by weight, and particularly preferably about 1 to about 40% by weight of polymerized, ethylenically unsaturated monomers which are copolymerizable with (α1), and

(α3) about 0.001 to about 10, preferably about 0.01 to about 7, % by weight and particularly preferably about 0.05 to about 5% by weight of one or more crosslinkers, the total of the amounts (α1) to (α3) being 100% by weight,

where the total of the % by weight of the components adds up to 100% by weight.

The monoethylenically unsaturated, acid-group-containing monomers (α1) can be partially or fully, preferably partially, neutralized. Preferably, the monoethylenically unsaturated, acid-group-containing monomers are neutralized to at least about 25 mol %, particularly preferably to at least about 50 mol %, and moreover preferably to about 50-90 mol %. Neutralization is preferably effected prior to polymerization. Furthermore, neutralization can be effected with alkali metal hydroxides, alkaline earth metal hydroxides, ammonia, carbonates and bicarbonates. In addition, any further base which forms a water-soluble salt with the acid is feasible. Mixed neutralization with a variety of bases is also feasible. Neutralization with ammonia or with alkali metal hydroxides, particularly preferably with sodium hydroxide or with ammonia, is preferred.

Preferred monoethylenically unsaturated, acid-group-containing monomers (α1) are acrylic acid, methacrylic acid, ethacrylic acid, α-chloroacrylic acid, α-cyanoacrylic acid, β-methylacrylic acid (crotonic acid), α-phenylacrylic acid, β-acryloxypropionic acid, sorbic acid, α-chlorosorbic acid, 2′-methylisocrotonic acid, cinnamic acid, p-chlorocinnamic acid, β-stearyl acid, itaconic acid, citraconic acid, mesaconic acid, glutaconic acid, aconitic acid, maleic acid, fumaric acid, tricarboxyethylene and maleic anhydride, with acrylic acid and methacrylic acid being particularly preferred and acrylic acid being very particularly preferred.

Other preferred monoethylenically unsaturated, acid-group-containing monomers (α1) are, besides the above carboxylate-group-containing monomers, ethylenically unsaturated sulfonic acid monomers or ethylenically unsaturated phosphonic acid monomers.

Ethylenically unsaturated sulfonic acid monomers are preferably aliphatic or aromatic vinylsulfonic acids or acrylic or methacrylic sulfonic acids. Preferred aliphatic or aromatic vinylsulfonic acids are vinylsulfonic acid, allylsulfonic acid, 4-vinylbenzyl-sulfonic acid, vinyltoluenesulfonic acid and styrenesulfonic acid. Preferred acryl- or methacrylsulfonic acids are sulfoethyl acrylate, sulfoethyl methacrylate, sulfopropyl acrylate, sulfopropyl methacrylate, 2-hydroxy-3-methacryloxypropylsulfonic acid and 2-acrylamido-2-methylpropanesulfonic acid.

Furthermore preferred are ethylenically unsaturated phosphonic acid monomers such as vinylphosphonic acid, allylphosphonic acid, vinzylbenzylphosphonic acid, acrylamidoalkylphosphonic acids, acrylamidoalkyldiphosphonic acids, phosphonomethylated vinyl amides and (meth)acrylphosphonic acid derivatives.

Preferred in accordance with the invention is that at least about 20 mol %, particularly preferably at least about 50 mol % and very particularly preferably at least about 75 mol % of the monoethylenically unsaturated, acid-group-containing monomers on which the polymer (A1) is based are carboxylate-group-containing monomers.

Preferred as ethylenically unsaturated monomers which contain a protinated nitrogen (α1) are dimethylaminoethyl (meth)acrylate hydrochloride and dimethylaminoethyl (meth)acrylate hydrogen sulfate.

Preferred as ethylenically unsaturated monomers containing a quaternized nitrogen (α1) are trimethylammonium ethyl (meth)acrylate methosulfate, trimethylammonium ethyl (meth)acrylate chloride, dimethylethylammonium ethyl (meth)acrylate ethosulfate, (meth)acrylamidopropyltrimethylammonium chloride and (meth)acrylamidopropyltrimethylammonium ethyl sulfate.

Preferred as monoethylenically unsaturated monomers (α2) which are capable of copolymerization with (α1) are acrylamides and methacrylamides.

Possible acrylamides are alkyl-substituted acrylamides or aminoalkyl-substituted derivatives of acrylamide or methylacrylamide, such as N-methylol(meth)acrylamide, vinylamides such as N-vinylamides, N-vinylformamides, N-vinylacetamides, N-vinyl-N-methylacetamides, N-vinyl-N-methylformamides, vinylpyrrolidone, N,N-dimethylaminoacrylamide, dimethylacrylamide or diethylacrylamide, acrylamidopropyltrimethylammonium chloride and the corresponding methacrylamide derivatives, and also acrylamide and methacrylamide, with acrylamide being preferred.

Furthermore preferred as monoethylenically unsaturated monomers (α2) which are capable of copolymerization with (α1) are water-dispersible monomers. Preferred as water-dispersible monomers are acrylic esters and methacrylic esters such as ethyl acrylate and ethyl methacrylate, butyl acrylate and butyl methacrylate, vinyl acetate, styrene and isobutylene.

Crosslinkers (α3) which are preferred in accordance with the invention are compounds which have at least two ethylenically unsaturated groups within a molecule (class I crosslinkers), compounds which have at least two functional groups which are capable of reacting with functional groups of the monomers (α1) or (α2) in a condensation reaction (class II crosslinkers), compounds which have at least one ethylenically unsaturated group and at least one functional group capable of reacting with functional groups of the monomers (α1) or (α2) (class III crosslinkers), or polyvalent metal cations (class IV crosslinkers). Here, crosslinking of the polymers is achieved by the class I crosslinker compounds by free-radical polymerization of the ethylenically unsaturated groups of the crosslinker molecule with the monoethylenically unsaturated monomers (α1) or (α2), while in the case of the class II crosslinker compounds and the polyvalent metal cations of the class IV crosslinkers crosslinking of the polymers is achieved by a condensation reaction of the functional groups (class II crosslinkers) or by electrostatic interaction of the polyvalent metal cation (class IV crosslinkers) with the functional groups of the monomers (α1) or (α2). In the case of the class III crosslinker compounds, accordingly, crosslinking of the polymer is effected both by free-radical polymerization of the ethylenically unsaturated group and by a condensation reaction between the functional group of the crosslinker and the functional groups of the monomers (α1) or (α2).

Preferred class I crosslinker compounds are poly(meth)acrylic esters which are obtained for example by reacting a polyol such as, for example, ethylene glycol, propylene glycol, trimethylolpropane, 1,6-hexanediol, glycerol, pentaerythritol, polyethylene glycol or polypropylene glycol, an amino alcohol, a polyalkylenepolyamine such as, for example, diethylenetriamine or triethylenetetraamine, or an alkoxylated polyol, with acrylic acid or methacrylic acid. Furthermore preferred as class I crosslinker compounds are polyvinyl compounds, poly(meth)allyl compounds, (meth)acrylic esters of a monovinyl compound or (meth)acrylic esters of a mono(meth)allyl compound, preferably of the mono(meth)allyl compounds of a polyol or an amino alcohol. Reference is made in this context to DE 195 43 366 and DE 195 43 368. The disclosures are herewith incorporated by reference and are thus considered as part of the disclosure.

Examples of class I crosslinker compounds which may be mentioned are alkenyl di(meth)acrylates such as, for example, ethylene glycol di(meth)acrylate, 1,3-propylene glycol di(meth)acrylate, 1,4-butylene glycol di(meth)acrylate, 1,3-butylene glycol di(meth)acrylate, 1,6-hexanediol di(meth)acrylate, 1,10-decanediol di(meth)acrylate, 1,12-dodecanediol di(meth)acrylate, 1,18-octadecanediol di(meth)acrylate, cyclopentanediol di(meth)acrylate, neopentyl glycol di(meth)acrylate, methylene di(meth)acrylate or pentaerythritol di(meth)acrylate, alkenyldi(meth)acrylamide, such as, for example, N-methyldi(meth)acrylamide, N,N′-3-methyl-butylidenebis(meth)acrylamide, N,N′-(1,2-dihydroxyethylene)bis(meth)acrylamide, N,N′-hexamethylenebis(meth)acrylamide or N′N′-methylenebis(meth)acrylamide, polyalkoxydi(meth)acrylates such as, for example, diethylene glycol di(meth)acrylate, triethylene glycol di(meth)acrylate, tetraethylene glycol di(meth)acrylate, dipropylene glycol di(meth)acrylate, tripropylene glycol di(meth)acrylate or tetrapropylene di(meth)acrylate, bisphenol A di(meth)acrylate, ethoxylated bisphenol A di(meth)acrylate, benzylidine di(meth)acrylate, 1,3-di(meth)acryloyloxypropan-2-ol, hydroquinone di(meth)acrylate, di(meth)acrylate ester of trimethylolpropane which has been alkoxylated, preferably ethoxylated, with preferably 1 to 30 moles of alkylene oxide per hydroxyl group, thioethylene glycol di(meth)acrylate, thiopropylene glycol di(meth)acrylate, thiopolyethylene glycol di(meth)acrylate, thiopolypropylene glycol di(meth)acrylate, divinyl ethers such as, for example, 1,4-butanediol divinyl ether, divinyl esters such as, for example, divinyl adipate, alkanedienes such as, for example, butadiene, divinylbenzene or 1,6-hexadiene, di(meth)allyl compounds such as, for example, di(meth)allyl phthalate or di(meth)allyl succinate, vinyl (meth)acrylic compounds such as, for example, vinyl (meth)acrylate, (meth)allyl (meth)acrylic compounds such as, for example, allyl (meth)acrylate, allyl (meth)acrylate which has been ethoxylated with 1 to 30 moles of ethylene oxide per hydroxyl group, di(meth)allyl esters of polycarboxylic acids such as, for example, di(meth)allyl maleate, di(meth)allyl fumarate, di(meth)allyl succinate or di(meth)allyl terephthalate, compounds with 3 or more ethylenically unsaturated groups capable of free-radical polymerization such as, for example, glycerol tri(meth)acrylate, (meth)acrylate esters of glycerol which has been ethoxylated with preferably 1 to 30 moles of ethylene oxide per hydroxyl group, trimethylolpropane tri(meth)acrylate, tri(meth)acrylic esters of trimethylolpropane which has been alkoxylated, preferably ethoxylated, with preferably 1 to 30 moles of alkylene oxide per hydroxyl group, trimethylacrylamide, (meth)allylidene di(meth)acrylate, 3-allyloxy-1,2-propanediol di(meth)acrylate, triallyl cyanurate, triallyl isocyanurate, pentaerythritol tetra(meth)acrylate, pentaerythritol tri(meth)acrylate, (meth)acrylic esters of pentaerythritol which has been ethoxylated with preferably 1 to 30 moles of ethylene oxide per hydroxyl group, tris(2-hydroxyethyl)isocyanurate tri(meth)acrylate, trivinyl trimellitate, triallylamine, triallylphosphate, tetraallylethylenediamine, polyallyl esters, tetraallyloxyethane or tetraallylammonium halides.

Particularly preferred among the crosslinkers of the class I crosslinkers are N,N′-methylenebisacrylamide, N,N′-methylenebismethacrylamide and triallylamine.

Preferred as class II crosslinker compounds are those which have at least two functional groups which are capable of reacting with the functional groups of the monomers (α1) or (α2), preferably with acid groups, of the monomers (α1) in a condensation reaction. These functional groups of the class II crosslinker compounds are preferably alcohol, amine, aldehyde, glycidyl, isocyanate, carbonate or epichlorine functions.

Examples of a class II crosslinker compound which may be mentioned are polyols such as, for example, ethylene glycol, polyethylene glycol such as diethylene glycol, triethylene glycol and tetraethylene glycol, propylene glycol, polypropylene glycols such as dipropylene glycol, tripropylene glycol or tetrapropylene glycol, 1,3-butanediol, 1,4-butanediol, 1,5-pentanediol, 2,4-pentanediol, 1,6-hexanediol, 2,5-hexanediol, glycerol, polyglycerol, trimethylolpropane, polyoxypropylene, oxyethylene/oxypropylene block copolymers, sorbitan fatty acid esters, polyoxyethylene sorbitan fatty acid esters, pentaerythritol, polyvinyl alcohol and sorbitol, amino alcohols such as, for example, ethanolamine, diethanolamine, triethanolamine or propanolamine, polyamine compounds such as, for example, ethylenediamine, diethylenetriamine, triethylenetetraamine, tetraethylenepentaamine or pentaethylenehexaamine, polyglycidyl ether compounds such as ethylene glycol diglycidyl ether, polyethylene glycol diglycidyl ether, glycerol diglycidyl ether, glycerol polyglycidyl ether, pentaerythritol polyglycidyl ether, propylene glycol diglycidyl ether, polypropylene glycol diglycidyl ether, neopentyl glycol diglycidyl ether, hexanediol glycidyl ether, trimethylolpropane polyglycidyl ether, sorbitol polyglycidyl ether, diglycidyl phthalate, adipic acid diglycidyl ether, 1,4-phenylenebis(2-oxazoline), glycidol, polyisocyanates, preferably diisocyanates such as 2,4-toluenediisocyanate and hexamethylenediisocyanate, polyaziridine compounds such as 2,2-bishydroxymethylbutanoltris[3-(1-aziridinyl)propionate], 1,6-hexamethylenediethyleneurea and diphenylmethanebis-4,4′-N,N′-diethyleneurea, halogen epoxides such as, for example, epichloro- and epibromohydrin and α-methyl-epichlorohydrin, alkylene carbonates such as 1,3-dioxolan-2-one, (ethylene carbonate), 4-methyl-1,3-dioxolan-2-one, (propylene carbonate), 4,5-dimethyl-1,3-dioxolan-2-one, 4,4-dimethyl-1,3-dioxolan-2-one, 4-ethyl-1,3-dioxolan-2-one, 4-hydroxymethyl-1,3-dioxolan-2-one, 1,3-dioxan-2-one, 4-methyl-1,3-dioxoan-2-one, 4,6-dimethyl-1,3-dioxan-2-one, 1,3-dioxolan-2-one, poly-1,3-dioxolan-2-one, polyquaternary amines such as condensates of dimethylamines and epichlorohydrin, homo- and copolymers of diallyldimethylammonium chloride and homo- and copolymers of diethylallylaminomethyl(meth)acrylate ammonium chloride. Furthermore preferred as class II crosslinker compounds are polyoxazolines such as 1,2-ethylenebisoxazoline, crosslinkers with silane groups such as γ-glycidoxypropyltrimethoxysilane and γ-aminopropyltrimethoxysilane, oxazolidinones such as 2-oxazolidinone, bis and poly-2-oxazolidinones and diglycol silicates.

Preferred as class III compounds are hydroxyl- or amino-group-containing (meth)acrylic acid esters, and N-substituted (meth)acrylamides.

Examples of possible hydroxyl- or amino-group-containing acrylic acid esters and hydroxyl- or amino-group-containing methacrylic acid esters are 2-hydroxyethyl acrylate, N,N-dimethylaminoethyl acrylate, and the analogous methacrylic acid derivatives in protonated or quaternized form such as, for example, dimethylaminoethyl (meth)acrylate hydrochloride, dimethylaminoethyl (meth)acrylic hydrogen sulfate, trimethylaminoethyl (meth)acrylate chloride, trimethylaminoethyl (meth)acrylate methosulfate or dimethylethylammonium methyl (meth)acrylate ethosulfate.

The polyvalent metal cations of the class IV crosslinkers are preferably derived from mono- or polyvalent cations, the monovalent ones in particular from alkali metals such as potassium, sodium and lithium, with lithium being preferred. Preferred divalent cations are derived from zinc, beryllium, alkaline earth metals such as magnesium, calcium and strontium, with magnesium being preferred. Further higher-valent cations which can be employed in accordance with the invention are cations of aluminum, iron, chromium, manganese, titanium, zirconium and other transition metals, and double salts of such cations or mixtures of the salts which have been mentioned. Substances which are preferably employed are aluminum salts and alums and their different hydrates such as, for example, AlCl₃×6H₂O, NaAl(SO₄)₂×12 H₂O, KAl(SO₄)₂×12 H₂O or Al₂(SO₄)₃×14-18 H₂O.

Class IV crosslinkers which are particularly preferably used are Al₂(SO₄)₃ and its hydrates.

Preferred embodiments of the water-in-oil polymer dispersion according to the invention are polymer dispersions in which the polymers are crosslinked by crosslinkers of the following classes of crosslinkers, or by crosslinkers of the following combinations of classes of crosslinkers: I, II, III, , III, III, IIV, III, II, I II IV, I III IV, II IV or III IV.

Further preferred embodiments of the water-in-oil polymer dispersion according to the invention are polymer dispersions in which the polymers are crosslinked by any one of the abovementioned crosslinkers from among the class I crosslinkers. Preferred among these are water-soluble crosslinkers. Particularly preferred in this context are N,N′-methylenebisacrylamide, polyethylene glycol diacrylates and dimethacrylates, triallylmethylammonium chloride, tetraallylammonium chloride, and allyl nonaethylene glycol acrylate made with 9 moles of ethylene oxide per mole of acrylic acid.

It is preferred in accordance with the invention that at least about 50 mol %, preferably at least about 90 mol % and particularly preferably at least about 99.9 mol % of the monomers in the polymer (A1) are water-soluble.

The hydrophobic organic fluid (A2) can be any hydrophobic organic fluid known to the skilled worker and employed as continuous phase in inverse emulsion polymerization. Preferred hydrophobic organic fluids (A2) are aromatic and aliphatic linear, branched and cyclic hydrocarbons. These include hydrocarbons or their mixtures, preferably n- and/or iso-paraffins, ligroin with a boiling range of from about 150 to about 200° C., in particular also distillates from predominantly paraffinic and naphthene-based mineral oils such as what are known as the white oils. Linear and branched liquid esters of natural and synthetic origin may furthermore be present as continuous phase. This includes the natural oils whose main constituents are predominantly triglycerides with a mono- or polyunsaturated carboxylic acid moiety derived from saturated C₁₀- to C₃₀-fatty acids, and the esters prepared therefrom with aliphatic alcohols. Furthermore preferred as hydrophobic organic fluid (A2) are those aliphatic dicarboxylic esters which are described in DE 35 24 950 A1, whose disclosure is herewith incorporated by reference. Mixtures of the abovementioned fluids may also be present as hydrophobic organic fluid (A2) in the water-in-oil polymer dispersions according to the invention.

Substances which may be present as water-in-oil emulsifiers (A3) in the water-in-oil polymer dispersion according to the invention are those compounds which are known to the skilled worker as water-in-oil emulsifiers which are employed in inverse emulsion polymerization. Preferred are water-in-oil emulsifiers (A3) with HLB values of from about 1 to about 10, preferably of from about 2 to about 8 and particularly preferably of from about 3 to about 5 (Römpps Chemie Lexikon [Römpp's dictionary of chemistry], 10th Edition, p. 1764, Franck'sche Verlagshandlung Stuttgart). Examples of preferred emulsifiers are sorbitan monostearate and sorbitan monooleate, fatty acid glycerides, di- and polyglycerol, sorbitol, sorbitan and adducts of alkylene oxides such as ethylene oxide and propylene oxide with higher linear and branched alcohols or alkylphenol. Furthermore preferred as water-in-oil emulsifiers are polymeric emulsifiers as are described, for example, in DE-C-2 4 12 266, whose disclosure is herewith incorporated as reference. Very particularly preferred emulsifiers are polymeric emulsifiers with the trade name Hypermer® (ICI, London, England). Others which may be present as water-in-oil emulsifiers (A3) in the water-in-oil polymer dispersion according to the invention are mixtures of different water-in-oil emulsifiers.

Substances which may be present as oil-in-water emulsifiers (A4) in the water-in-oil polymer dispersion according to the invention are the compounds known to the skilled worker as inverters or activators which are employed in water-in-oil polymer dispersions. Preferred are ethoxylated fatty alcohols, ethoxylated fatty alcohols prepared from linear and/or branched fatty alcohols with an alkyl chain length of more than 11 carbon atoms being particularly preferred. Likewise preferred are ethoxylates of highly-branched alcohols which are obtainable by oxo synthesis, such as, for example, isotridecyl alcohol. Particularly preferred as inverters are ethoxylates of higher singly-branched alcohols which are obtainable by Guerbet synthesis. Mixtures of various oil-in-water emulsifiers may also be present as oil-in-water emulsifiers (A4) in the water-in-oil polymer dispersion according to the invention.

Auxiliaries (A5) which may be present in the water-in-oil polymer dispersions according to the invention are the compounds for complexing extraneous ions such as, for example, Versenex® 80, chain terminators such as, for example, formic acid, wetters, spreading agents, antifreeze agents, colorants and adhesion promoters, all of which are known to the skilled worker.

In a preferred embodiment of the composition according to the invention, the latter can be poured through a screen of mesh size 150 μm at 20° C. and a pressure of 1 bar, without particles >150 μm which comprise the crosslinked polymer remaining on the screen.

In a particularly preferred embodiment of the composition according to the invention, the latter can be poured through a screen of mesh size 90 μm at 20° C. and a pressure of 1 bar, without particles >90 μm which comprise the crosslinked polymer remaining on the screen.

The invention furthermore relates to a process for the preparation of the composition according to the invention, wherein water is mixed with the additive in the form of a water-in-oil polymer dispersion (PD), which comprises a hydrophobic organic phase and crosslinked polymers distributed therein and is based on

(PD1) a crosslinked polymer in an amount in a range of about 10 to about 70% by weight, preferably in a range of from about 20 to about 50% by weight and very particularly preferred in a range of from about 25 to about 35% by weight, in each case based on the total weight of the additive,

(PD2) a hydrophobic organic fluid in an amount in a range of from about 20 to about 80% by weight, preferably in a range of from about 25 to about 50% by weight, and very particularly preferably in a range of from about 30 to about 40% by weight, in each case based on the total weight of the additive,

(PD3) one or more water-in-oil emulsifiers in an amount in a range of from about 0.5 to about 10% by weight, preferably in a range of from about 1 to about 8% by weight, and very particularly preferably in a range of from about 2 to about 6% by weight, in each case based on the total weight of the additive,

(PD4) one or more oil-in-water emulsifiers in an amount in a range of from about 0.5 to about 10% by weight, preferably in a range of from about 1 to about 8% by weight, and very particularly preferably in a range of from about 2 to about 6% by weight, in each case based on the total weight of the additive,

(PD5) one or more auxiliaries in an amount of in a range of from 0 to about 20% by weight, preferably in a range of from about 0.01 to about 10% by weight and very particularly preferably in a range of from about 0.1 to about 5% by weight, in each case based on the total weight of the additive, and

(PD6) water in an amount of at least about 0.1% by weight, preferably at least about 1% by weight and particularly preferably at least about 5% by weight, in each case based on the total weight of the additive, but at least in such an amount that the total of (PD1) to (PD6) amounts to 100% by weight,

and with at least one active ingredient which differs from the water-in-oil polymer dispersion (PD).

The active ingredient which differs from the water-in-oil polymer dispersion (PD), the crosslinked polymer (PD1), the hydrophobic, organic fluid (PD2), the water-in-oil emulsifier (PD3), the oil-in-water emulsifier (PD4) and the auxiliaries (PD5) preferably correspond to the active ingredients (A), crosslinked polymers (A1), hydrophobic, organic fluids (A2), water-in-oil emulsifiers (A3), oil-in-water emulsifiers (A4) and auxiliaries (A5) which have been mentioned at the outset in connection with the composition according to the invention. Tap water is preferably used as water (C) for the composition according to the invention, while deionized water is preferably used as water (PD6) in the water-in-oil polymer dispersion.

The water-in-oil polymer dispersion is prepared by the emulsion polymerization processes which are known to the skilled worker, preferably by inverse emulsion polymerization. Preferably, the monomers (α1), (α2) and, if appropriate, the crosslinkers (α3) and the auxiliaries (PD5) are dissolved in water (PD6) and this solution is then dispersed in the hydrophobic, organic fluid (PD2) in the presence of one or more water-in-oil emulsifiers (PD3). After the monomer emulsion has been purged with nitrogen for approximately 1-2 hours with stirring, the polymerization is started by adding polymerization initiators. It is also possible to disperse the polymerization initiators in the composition before adding the hydrophobic, organic fluid (PD2) in order to ensure uniform distribution of the initiators in the composition.

Polymerization initiators may be present in a solution of monomers according to the invention in dissolved or dispersed form. Suitable initiators are all of the compounds which break down into free radicals and which are known to the skilled worker. This includes, in particular, peroxides, hydroperoxides, hydrogen peroxide, persulfates, azo compounds and what are known as redox catalysts. The use of water-soluble catalysts is preferred. In some cases it is advantageous to use mixtures of various polymerization initiators. Preferred among these mixtures are mixtures of hydrogen peroxide and sodium or potassium peroxodisulfate, which can be employed in any feasible proportion. Organic peroxides which are preferably suitable are acetylacetone peroxide, methyl ethyl ketone peroxide, t-butyl hydroperoxide, cumene hydroperoxide, t-amyl perpivate, t-butyl perviate, t-butyl pemeohexonate, t-butyl isobutyrate, t-butyl per-2-ethylhexenoate, t-butyl perisononanoate, t-butyl permaleate, t-butyl perbenzoate, t-butyl 3,5,5-trimethylhexanoate and amyl pemeodecanoate. Others which are preferred as polymerization initiators are: azo compounds such as 2,2′-azobis(2-amidinopropane) dihydrochloride, azobisamidinopropane dihydrochloride, 2,2′-azobis(N,N-dimethylene)isobutyramidine dihydrochloride, 2-(carbamoylazo)isobutyronitrile and 4,4′-azobis(4-cyanovaleric acid). The stated compounds are employed in customary amounts, preferably in a range of from about 0.01 to about 5, preferably from about 0.1 to about 2 mol %, in each case based on the amount of the monomers to be polymerized.

The redox catalysts comprise at least one of the abovementioned per compounds as oxidic component and preferably ascorbic acid, glucose, sorbose, mannose, ammonium hydrogen sulfite, ammonium hydrogen sulfate, ammonium thiosulfate, ammonium hyposulfite, ammonium sulfide, alkali metal hydrogen sulfite, alkali metal sulfate, alkali metal thiosulfate, alkali metal hyposulfite, alkali metal sulfide, metal salts such as iron(II) ions or silver ions or sodium hydroxymethylsulfoxylate as reducing component. Ascorbic acid or sodium pyrosulfite is preferably used as reducing component of the redox catalyst. About 1×10⁻⁵ to about 1 mol % of the reducing component of the redox catalyst and about 1×10⁻⁵ to about 5 mol % of the oxidizing component of the redox catalyst are employed based on the amount of monomers employed in the polymerization. One or more azo compounds, preferably water-soluble azo compounds, may be used instead of the oxidizing component of the redox catalyst or in conjunction with it.

A redox system composed of hydrogen peroxide, sodium peroxidisulfate and ascorbic acid is preferably employed in accordance with the invention. In general, azo compounds are preferred in accordance with the invention as initiators, with azobisamidinopropane dihydrochloride being particularly preferred. As a rule, the polymerization is initiated with the initiators in a temperature range of from about 30 to about 90° C.

The polymerization can be carried out as an isothermic process, an adiabatic process or a combination of an isothermic and adiabatic process.

Preferably, the polymerization is carried out as an isothermic process. Here, the polymerization process is started at a particular temperature under reduced pressure, as described for example in EP 228 397 B1, whose disclosure is herewith incorporated as reference. The reduced pressure is adjusted in such a way that, owing to the resulting heat of polymerization, volatile substances such as water and constituents of the organic phase can be distilled off and the temperature can be kept virtually constant. The end of the polymerization is characterized in that there is no more passing over of the distillate.

In the case of adiabatic polymerization, the polymerization process is started up at a specific temperature in the range of from 0 to about 50° C., preferably 0 to about about 25° C. However, the polymerization is carried out under atmospheric pressure without external heating until a maximum final temperature of the dispersion is reached, owing to the heat of polymerization, which depends on the content polymerizable substance of in the dispersion.

If the polymerization process is carried out as a combination of an isothermic and adiabatic process, the process is preferably first carried out as an isothermic process. The apparatus is aerated with inert gas at a previously determined point in time, and polymerization is continued until a particular final temperature has been reached.

After the polymerization, the water-in-oil polymer dispersion is cooled. Finally, the oil-in-water emulsifiers (PD4) are added to the water-in-oil polymer dispersion as activators or inverters.

The water-in-oil polymer dispersion used in the process according to the invention preferably has polymer particles with a size of less than about 10 μm, preferably less than about 2 μm and very particularly preferred of less than about 1 μm.

Moreover, it is preferred that the additive in an aqueous mixture consisting of water and 2.75% by weight of the water-in-oil polymer dispersion (PD), based on the total weight of the aqueous mixture, where the water-in-oil polymer dispersion (PD) comprises 28% by weight of the crosslinked polymer (PD1), based on the total weight of the water-in-oil polymer dispersion (PD), has at least one of the following characteristics:

(α) a viscosity of at least 5000 mPa·s, preferably of at least 10,000 mPa·s and very particularly preferred of at least 15,000 mpa·s at 4 revolutions per minute,

(β) a viscosity of at least 500 mPa·s, preferably of at least 1000 mpa·s, very particularly preferred of at least 2000 mPa·s at 128 revolutions per minute.

The combinations, resulting from the above characteristics (α) to (β), of two or more of these characteristics are in each case preferred embodiments of the water-in-oil polymer dispersions employed in the method according to the invention. Furthermore particularly preferred as embodiments according to the invention are methods in which water-in-oil polymer dispersions with the characteristics, or combinations of characteristics, represented hereinbelow as letters or combinations of letters: α, β, αβ.

Mixing of the water-in-oil polymer dispersion (PD), the water and the active ingredient is carried out by simply combining and mixing the individual components. In this context, it is possible first to mix the water-in-oil polymer dispersion with the water and subsequently to add the active ingredient. However, it is also possible to add the active ingredient as early as before or during mixing of the water with the water-in-oil polymer dispersion. Preferably, the active ingredient of the composition according to the invention is added after the additive has been mixed with the water.

Mixing of the water-in-oil polymer dispersion with water or with water comprising an active ingredient is preferably carried out by conveying the aqueous phase with the water-in-oil polymer dispersion (PD) toward a shearing tool, preferably a static mixer. The aqueous phase with the water-in-oil polymer dispersion (PD) is conveyed to this shearing tool at a pressure of greater than about 1.1, preferably greater than about 1.5 and particularly preferably greater than about 2 bar, it furthermore being preferred for the pressure not to exceed about 10 bar.

In an embodiment of the method according to the invention, the active ingredients can be mixed in pure form with the water and the additive.

In another embodiment of the method according to the invention, the active ingredients can be mixed with the water and the additive in the form of active ingredient compositions comprising the active ingredient and formulation auxiliaries such as, for example, organic solvents.

Preferred embodiments of the method according to the invention are those in which at least one of the active ingredients mentioned in connection with the compositions according to the invention, but not tebuconazole, imidacloprid, thiacloprid, trifloxy-strobin and iprovalicarb, is employed. In another embodiment of the method which is preferred in accordance with the invention at least one of the active ingredients tebuconazole, imidacloprid, thiacloprid, trifloxystrobin and iprovalicarb, preferably all of the abovementioned active ingredients, are employed.

The invention furthermore relates to a composition which can be obtained by the method according to the invention. Preferably, this composition has the same characteristics as the composition according to the invention which has been described at the outset.

The invention furthermore relates to the use of the additive (A) or an additive in the form of the water-in-oil polymer dispersion (PD) in a composition comprising at least one active ingredient which differs from the additive for

(β1) increasing the efficacy of the active ingredient in comparison with the same dose of active ingredient without the additive (A) under otherwise identical conditions when controlling an organism; or

(β2) reducing the decrease in efficacy of the active ingredient as a function of time in comparison with the same dose of active ingredient without the additive (A) under otherwise identical conditions when controlling an organism.

Preferred embodiments of the use according to the invention are those uses which result from the following letters or combinations of letters: β1, β2 and β1β2.

Preferably, the efficacy of the active ingredient in controlling an organism (β1) is the efficacy defined by Abbott, which is determined by the equation hereinbelow and stated in %:

$$Efficacy as defined by Abbott = 100 \cdot \{ 1 - \frac{\% disease ( treated substrate )}{disease ( untreated substrate )} \}$$

When controlling an organism, the efficacy is increased when the efficacy when controlling the organism in the presence of the additive exceeds the efficacy when controlling an organism in the absence of the additive.

The invention furthermore relates to the use of the additive according to the invention in a composition encompassing an active ingredient combination comprising at least two different active ingredients which differ from the additive for increasing the efficacy of at least one combination of two of the active ingredients (X, Y) of the active ingredient combination when controlling an organism in comparison with the efficacy W(syn), where

$$W ( s y n ) = W ( X ) + W ( Y ) - \{ \frac{W ( X ) \cdot W ( Y )}{100} \}$$

applies to W(syn) and where, in the above equation,

W(X) is the efficacy of the active ingredient X when applying the same amount of the active ingredient X as in the active ingredient combination in the presence of the additive and

W(Y) is the efficacy of the active ingredient Y when applying the same amount of the active ingredient Y as in the active ingredient combination in the presence of the additive,

and where the ratio of active ingredient to additive in the active ingredients and the active ingredient combination is the same in each case (=synergistic effect).

Preferred as active ingredients for the uses (β1) and (β2) or as active ingredients in the active ingredient combination for controlling an organism are those active ingredients which are capable of controlling organisms and which have been mentioned in connection with the composition according to the invention.

Organisms are preferably harmful plants, in particular broad-leafed weeds and grass weeds, fungal pests, animal pests such as, for example, insect pests, harmful bacteria, harmful nematodes and harmful viruses.

The organisms which are controlled with the additive according to the invention are preferably located on or in a substrate. Preferred substrates are intact plants or plant parts such as stems, branches, flowers, leaves, intact or divided roots or tubers, seed kernels, seeds or fruit, smooth or rough surfaces such as walls, car tires or soils, liquid substrates such as, for example, water, in particular water, or gaseous substrates such as, for example, air.

The invention furthermore relates to the use of the additive (A) or an additive in the form of the water-in-oil polymer dispersion (PD) in a composition comprising at least one active ingredient which differs from the additive for

(γ1) increasing the efficacy of the active ingredient in comparison with the same dose of active ingredient without the additive (A) under otherwise identical conditions when regulating the growth of a plant; or

(γ2) reducing the decrease in efficacy of the active ingredient as a function of time in comparison with the same dose of active ingredient without the additive (A) under otherwise identical conditions when regulating the growth of a plant.

Preferred embodiments of the use according to the invention are those uses which result from the following letters or combinations of letters: γ1, γ2 and γ1γ2.

The efficacy of the active ingredient when regulating the growth of a plant (γ1) is preferably understood as meaning a plant growth quotient. The plant growth quotient is defined as follows:

$$Plant growth quotient = 100 \cdot \{ 1 - \frac{growth level ( treated plant )}{growth level ( untreated plant )} \}$$

In regulating the growth of a plant, the efficacy is increased when the plant growth quotient in regulating the growth in the presence of the additive exceeds the efficacy when regulating the growth in the absence of the additive.

The invention furthermore relates to the use of the additive according to the invention in a composition encompassing an active ingredient combination comprising at least two different active ingredients which differ from the additive for increasing the efficacy of at least one combination of two of the active ingredients (X, Y) of the active ingredient combination in regulating the growth of a plant in comparison with the efficacy W(syn), where

$$W ( s y n ) = W ( X ) + W ( Y ) - \{ \frac{W ( X ) \cdot W ( Y )}{100} \}$$

applies to W(syn) and where, in the above equation,

W(X) is the efficacy of the active ingredient X when applying the same amount of the active ingredient X as in the active ingredient combination in the presence of the additive and

W(Y) is the efficacy of the active ingredient Y when applying the same amount of the active ingredient Y as in the active ingredient combination in the presence of the additive,

and where the ratio of active ingredient to additive in the active ingredients and the active ingredient combination is the same in each case (=synergistic effect).

Preferred as active ingredients for the uses (γ1) and (γ2) or as active ingredients for use in an active ingredient combination for regulating the growth of a plant are those active ingredients which are capable of regulating the growth of a plant and which have been mentioned in connection with the composition according to the invention.

Furthermore, the invention relates to a method for controlling an organism wherein the organism is brought into contact with the compositions according to the invention.

In the method according to the invention for controlling an organism, the organism is preferably located on or in a substrate.

Preferred substrates and organisms are those substrates or organisms which have already been mentioned in connection with the uses (β1) and (β2) according to the invention.

The invention furthermore relates to a method for regulating the growth of a plant, wherein the plant is brought into contact with the compositions according to the invention.

Bringing-into-contact with the compositions according to the invention is preferably effected by brushing on, immersing or spraying on, spraying on being particularly preferred.

The compositions according to the invention are preferably sprayed on from a spray mix tank.

The invention furthermore relates to the use of the compositions according to the invention in agriculture, in forestry, in horticulture, in fruit production, in the control of vectors, in plant growing, in plant breeding, in connection with seed, plant materials, nonagricultural applications, for controlling or combating organisms, and in connection with the storage or processing of fruits and crops or plant materials.

Particularly preferred is the use of the compositions according to the invention as seed treatment product and in the treatment of plant materials, preferably roots or tubers.

The invention is subsequently illustrated in greater detail with reference to test methods and nonlimiting examples.

### TEST METHODS

### Flow Rate Reduction

Using a pipette, 1.0 g of the composition is placed centrally (8 cm from the side edge) at a height of 20 cm on a sheet of plexiglass (length 46 cm, width 16 cm). The sheet is arranged at a slant on a tabletop so that the angle between the plane tabletop and the sheet of plastic is 20°. The distance which the mixture has traveled is monitored over time. The flow rate is stated in cm/sec and is calculated using the following equation:

$$Flowrate = \frac{the distance traveled by the aqueous phase}{time}$$

### Prolongation of the Water Evaporation Period

The prolongation of the period which elapses until 100% by weight of the water of the composition have evaporated at 22° C. and a pressure of 1 bar was calculated by weighing 10 g of the composition into a crystallization dish of 9 cm diameter and distributing it uniformly at the bottom of the dish. The dishes were left to stand uncovered in a room at a temperature of 22° C. and a pressure of 1 bar. The weight loss was determined every hour or every half hour. A 100% evaporation of the water is reached when the weight of the crystallization dish remains constant over a period of 10 hours.

### Determination of the Viscosity of the Water-in-oil Polymer Dispersion

The viscosity of the water-in-oil polymer dispersion was determined using an RV 20 Rotovisco viscometer from Haake equipped with an M5 measuring head.

972.5 g water with a hardness of 20-25° dH are introduced into a 2 l plastic beaker, and 27.5 g of product are then added using a disposable syringe. The mixture is stirred for 5 minutes at 3200 revolutions per minute using the Mutifix-Record stirrer and left to stand for 10 minutes.

The Haake measuring beaker is filled with the solution produced in such a way that the uppermost mark located in the beaker is covered by the solution. The measuring beaker is now introduced into the Haake viscometer which had previously been zeroed and equipped with the measuring unit. The measurements were carried out at settings 3 and 8. The reading shown is multiplied with the factor which corresponds to each setting, as can be seen from the table which follows.

| Setting | Revolutions per minute | Factor |
|---|---|---|
| 3 | 4 | 3077 |
| 8 | 128 | 96 |

### Determination of the Size of the Polymer Particles in the Water-in-oil Polymer Dispersion

10 μl of the water-in-oil polymer dispersion are pipetted into a microscopic slide and covered with a cover glass. The size of the polymer particles is established under the light microscope as the mean size of 100 polymer particles.

### Increasing the Efficacy of the Active Ingredient, and Reducing the Decrease of the Efficacy, as a Function of Time when Controlling an Organism

In a stand of winter wheat cv. “Haven”, which had been sown in November, plots were sprayed with the composition during growth stage 37 (appearance of pointed flag leaf) and at growth stage 47 (flag leaf sheath splits) (growth stages of cereals: Novartis Produktinformation 2000, Novartis Agro GmbH). The compositions were prepared in such a way that, initially, in each case, an amount of a water-in-oil polymer dispersion which is defined for a hectare is stirred into 300 liters of water (spray water quantity), and the commercial active ingredient products (active ingredient formulations) were subsequently stirred in. Randomly distributed test plots (1.5 m×5 m=7.5 m²) in the trial field were sprayed in four replications with the respective spray mixture quantity (300 liters/hectare) (compositions without additive acted as controls). A knapsack sprayer with a spray boom of 1.5 m working width and with 6 Universal fan nozzles (Lechler 90-02) spaced 25 cm apart were used to apply the spray mixture. The infection of the wheat leaves (10 per plant) with the plant disease Septoria tritici was scored at various points in time after the application of the compositions. The increase in efficacy was determined after 22 days and after 35 days by the method of Abbott, using the following formula:

$$Efficacy as defined by Abbott = 100 \cdot \{ \frac{\% disease level ( treated plant )}{\% disease level ( untreated plant )} \}$$

### Increasing the Efficacy of the Active Ingredient, and Reducing the Decrease of the Efficacy, as a Function of Time in Regulating the Growth of a Plant

In a stand of triticale, plots were sprayed with the composition at growth stage 39 (in which the flag leaf is fully developed) (growth stages of cereals: Novartis Produktinformation 2000, Novartis Agro GmbH). The compositions were prepared in such a way that, initially, in each case, an amount of a water-in-oil polymer dispersion which is defined for a hectare is stirred into 300 liters of water (spray water quantity), and the commercial growth regulators were subsequently stirred in. Randomly distributed test plots (1.5 m×5 m=7.5 m²) in the trial field were sprayed in four replications with the respective spray mixture quantity (300 liters/hectare) (compositions without additive acted as controls). A knapsack sprayer with a spray boom of 1.5 m working width and with 6 Universal fan nozzles (Lechler 90-02) spaced 25 cm apart were used to apply the spray mixture. 14 days and 28 days after the application, the plant height in the plots was measured from the soil to mid-ear. The efficacy of the active ingredient in regulating the growth of the plant is indicated by the plant growth quotient.

### EXAMPLES

The additive employed in the examples which follow is the water-in-oil polymer dispersion Firesorb® MO from Stockhausen GmbH & Co. KG.

### Example 1

Effect of the water-in-oil polymer dispersion on the efficacy of Desmel (by Syngenta, 250 g propiconazole/l) in the control of Septoria tritici in wheat:

Wheat was sprayed with a composition comprising the amounts of the water-in-oil polymer dispersion stated in Table 1 and Desmel in the stated amounts. 22 days after spraying, the disease level of the wheat leaves on the leaf underneath the flag leaf was scored. The following data were recorded for the increase in the efficacy of the active ingredient propiconazole:

TABLE 1

| | Amount of product | Amount of additive | Activity, found |
|---|---|---|---|
| Product | (l/ha) | (l/ha) | (%) |
| Desmel | 0.5 | 0 | 66 |
| Desmel | 0.5 | 0.5 | 78 |
| Desmel | 0.5 | 1 | 95 |
| Desmel | 0.5 | 2 | 88 |
| Without | 0 | 1 | 0 |

The infection level of the untreated controls was 12%.

It can be seen from Table 1 that the additive (water-in-oil polymer dispersion) increases the efficacy of the product.

### Example 2

Effect of the water-in-oil polymer dispersion on the efficacy of the active ingredient combination Opus Top (from BASF, comprises the active ingredient components epoxiconazole and fenpropimorph in an amount of 84 g/l and 250 g/l, respectively) in the control of Septoria tritici in wheat:

Wheat was sprayed with a composition comprising the amounts of the water-in-oil polymer dispersion stated in Table 2 and Opus Top in the stated amounts. 22 days and 35 days after spraying, the disease level of the wheat leaves on the leaf underneath the flag leaf was scored. The following data were recorded for the increase in efficacy of the active ingredient combination epoxiconazole and fenpropimorph:

TABLE 2

| | Amount | Amount | | | Activity |
|---|---|---|---|---|---|
| | of | of | Activity | Activity | expected |
| | product | additive | found after | found after | after |
| Product | (l/ha) | (l/ha) | 22 days (%) | 35 days (%) | 22 days (%) |
| Opus Top | 0.75 | 0 | 91 | 57% | |
| Opus Top | 0.75 | 1 | 98 | 84% | 91 + 0 = 91 |
| Without | 0 | 1 | 0 | | |

The infection level of the untreated controls were 16% after 22 days and 81% after 35 days.

It can be seen from Table 2 that the additive (water-in-oil polymer dispersion) increases the efficacy of the product. The effect is synergistic since the activity found exceeds the activity which was expected by adding the activities of the individual active ingredient components.

It can also be seen from the table that the additive reduces the decrease in efficacy of the product within a period of 13 days (35 days-22 days).

### Example 3

Effect of the water-in-oil polymer dispersion on the efficacy of the active ingredient combination Gladio (from Syngenta, comprises the active ingredient components tebuconazole, 125 g/l, propiconazole, 125 g/l and fenpropidin, 375 g/l) in the control of Septoria tritici in wheat:

Wheat was sprayed with a composition comprising the amounts of the water-in-oil polymer dispersion stated in Table 3 and Gladio in the stated amounts. 22 days after spraying, the disease level of the wheat leaves on the leaf underneath the flag leaf was scored. The following data were recorded for the increase in efficacy of the active ingredient combination tebuconazole, propiconazole and fenpropidin:

TABLE 3

| | Amount of | Amount of | | Activity |
|---|---|---|---|---|
| | product | additive | Activity found | expected |
| Product | (l/ha) | (l/ha) | (%) | (%) |
| Gladio | 0.4 | 0 | 80 | |
| Gladio | 0.4 | 1 | 92 | 80 + 0 = 80 |
| Without | 0 | 1 | 0 | |

The disease level of the untreated controls was 21%.

It can be seen from Table 3 that the additive (water-in-oil polymer dispersion) increases the efficacy of the product. The effect is synergistic since the activity found exceeds the activity which was expected by adding the activities of the individual active ingredient components.

### Example 4

The effect of additive on the stem-shortening effect of Cycocel (CCC) (from BASF, contains the growth regulator chlormequat chloride, 558 g/l) in the treatment of triticale:

Triticale was sprayed with a composition comprising the amounts of the water-in-oil polymer dispersion stated in Table 4 and Cycocel in the stated amounts. 14 days and 27 days after the application, plant lengths was determined. The following data were recorded for the plant growth quotient and thus for the efficacy of the active ingredient chlormequat chloride in the treatment of triticale:

TABLE 4

| | Amount of | Amount of | | |
|---|---|---|---|---|
| | product | additive | Activity after | Activity after |
| Product | (l/ha) | (l/ha) | 14 days (%) | 27 days (%) |
| CCC | 2 | 0 | 6.8 | 10.6 |
| CCC | 1 | 0 | 3.9 | 7.2 |
| CCC | 1 | 1 | 6.3 | 11.9 |
| Without | 0 | 0 | 0 | 0 |

It can be seen from Table 4 that the additive (water-in-oil polymer dispersion) increases the stem-shortening effect of the product.

It can also be seen from Table 4 that the additive reduces the decline in efficacy of the active ingredient within a period of 13 days (27 days-14 days).

### Example 5

The effect of additive on the stem-shortening effect of Moddus (from BASF, contains the growth regulator trinexapec, 222 g/l) in the treatment of triticale:

Triticale was sprayed with a composition comprising the amounts of the water-in-oil polymer dispersion stated in Table 5 and Moddus in the stated amounts. 14 days and 27 days after the application, plant lengths was determined. The following data were recorded for the plant growth quotient and thus for the efficacy of the active ingredient trinexapec in the treatment of triticale:

TABLE 5

| | Amount of | Amount of | | |
|---|---|---|---|---|
| | product | additive | Activity after | Activity after |
| Product | (l/ha) | (l/ha) | 14 days (%) | 27 days (%) |
| Moddus | 0.6 | 0 | 13.9 | 12.5 |
| Moddus | 0.3 | 0 | 5.6 | 7.0 |
| Moddus | 0.3 | 1 | 8.3 | 10.8 |
| Without | 0 | 0 | 0 | 0 |

It can be seen from Table 5 that the additive (water-in-oil polymer dispersion) increases the stem-shortening effect of the product.

It can also be seen from Table 5 that the additive reduces the decline in efficacy of the active ingredient within a period of 13 days (27 days-14 days).

### Example 6

Determination of the synergistic effect of the additive on an active ingredient combination by CCC (from BASF, contains the growth regulator chlormequat chloride, 558 g/l) and Moddus (from BASF, contains the growth regulator trinexapec, 222 g/l). The synergistic effects were determined with the aid of what is known as the Colby formula (Colby, S. R. (1967): “Calculating Synergistic and Antagonistic Responses of Herbicide Combinations”, Weeds, 15: p. 20-22).

Triticale was sprayed with a composition comprising the amounts of the water-in-oil polymer dispersion stated in Table 6 and Moddus and/or CCC in the stated amounts. 14 days after the application, plant length was determined. The following data were determined for the plant growth quotient and thus for the efficacy of the active ingredients trinexapec and/or chlormequat chloride in the treatment of triticale:

TABLE 6

| | Amount of | Amount of | | |
|---|---|---|---|---|
| | product | additive | Activity after | Expected activity after 14 |
| Product | (l/ha) | (l/ha) | 14 days (%) | days (%) |
| CCC | 1 | 1 | 6.3 | |
| Moddus | 0.3 | 1 | 8.3 | |
| CCC + | 1 + | 1 | 21.8 | 6.3 + 8.3 − |
| Moddus | 0.3 | | | (6.3 × 8.3)/100 = 14 |

### Example 7

Effect of the additive on reducing evaporation:

The water-in-oil polymer dispersion was mixed with water in the amounts stated in Table 7 hereinbelow. Tap water without additive acted as control.

TABLE 7

| | Time [h] elapsed | Time [h] elapsed |
|---|---|---|
| | until 95% by | until 100% by |
| | weight of the water | weight of the water |
| Composition | had evaporated | had evaporated |
| Tap water | 19 | 21 |
| Tap water + 0.5% by weight | 20.5 | 24 |
| of the additive | | |
| Tap water + 1.0% by weight | 20.5 | 24 |
| of the additive | | |
| Tap water + 1.5% by weight | 21.5 | 27 |
| of the additive | | |
| Tap water + 2.0% by weight | 20.5 | 26 |
| of the additive | | |

It can be seen from Table 7 that the addition of the additive, in the amounts stated in Table 7, to an aqueous mixture extends the period of time which elapses until a given amount of the water present in the aqueous mixture (100% by weight and 95% by weight, respectively) evaporates in comparison with the same amount of pure tap water.

### Example 8

Effect of the additive on reducing the flow rate of the composition:

The water-in-oil polymer dispersion was mixed with water in the amounts stated in

Table 8 hereinbelow. Tap water without additive acted as control.

TABLE 8

| | | Amount of mixture still |
|---|---|---|
| | Flow rate | remaining on the sheet |
| Composition | [cm/sec] | after 5 minutes [g] |
| Tap water | 20 | 0.2 |
| Tap water + 0.5% by weight of the | 10 | 0.1 |
| additive | | |
| Tap water + 1.0% by weight of the | 1 | 0.2 |
| additive | | |
| Tap water + 1.5% by weight of the | 0.11 | 0.7 |
| additive | | |
| Tap water + 2.0% by weight of the | <0.0027 | 1.0 |
| additive | | |

It can be seen from Table 8 that the addition of the additive, in the amounts stated in Table 8, to an aqueous mixture reduces the flow rate.

The above description of the invention is intended to be illustration and not limiting. Various changes or modifications in the embodiments may occur to those skilled in the art. These can be made without departing from the scope or spirit of the invention.

1. A composition comprising
(A) an additive in an amount in a range of from about 0.001 to about 2% by weight, based on the total weight of the composition,
(B) at least one active ingredient which differs from the additive (A), and
(C) water in an amount of at least about 50% by weight, based on the total weight of the composition,
where the additive (A) comprises
(A1) a crosslinked polymer in an amount in a range of from about 10 to about 70% by weight, based on the total weight of the additive (A),
(A2) a hydrophobic organic fluid in an amount in the range of from about 20 to about 80% by weight, based on the total weight of the additive (A),
(A3) one or more water-in-oil emulsifiers in an amount in a range of from about 0.5 to about 10% by weight, based on the total weight of the additive (A),
(A4) one or more oil-in-water emulsifiers in an amount in a range of from about 0.5 to about 10% by weight, based on the total weight of the additive (A),
(A5) one or more auxiliaries in an amount in a range of from 0 to about 20% by weight, based on the total weight of the additive (A), and
(A6) water in such an amount that the total of (A1) to (A6) amounts to 100% by weight.

2. The composition as claimed in claim 1, wherein the composition is pourable at 20° C. and a pressure of 1 bar through a screen of mesh size 150 μm without particles >150 μm comprising the crosslinked polymer remaining on the screen.

3. The composition as claimed in claim 1 comprising
(A) an additive in an amount in a range of from 0.001 to 2% by weight, based on the total weight of the composition,
(B) at least one active ingredient which differs from the additive (A), and
(C) water in an amount of at least 50% by weight, based on the total weight of the composition,
where the additive (A) comprises
(A1) a crosslinked polymer in an amount in a range of from 10 to 70% by weight, based on the total weight of the additive (A),
(A2) a hydrophobic organic fluid in an amount in the range of from 20 to 80% by weight, based on the total weight of the additive (A),
(A3) one or more water-in-oil emulsifiers in an amount in a range of from 0.5 to 10% by weight, based on the total weight of the additive (A),
(A4) one or more oil-in-water emulsifiers in an amount in a range of from 0.5 to 10% by weight, based on the total weight of the additive (A),
(A5) one or more auxiliaries in an amount in a range of from 0 to 20% by weight, based on the total weight of the additive (A), and
(A6) water in such an amount that the total of (A1) to (A6) amounts to 100% by weight.

4. A composition as claimed in claim 1, wherein the additive (A) in a composition composed of water and the additive in an amount of 1.5% by weight, based on the total weight of the composition, has at least one of the following characteristics:
(a1) reduced flow rate of the aqueous mixture by at least 75%, in each case in comparison with the flow rate of water;
(a2) extended period of time which elapses until the water present in 10 g of the composition has evaporated at 22° C. and a pressure of 1 bar in comparison with the period of time which elapses until the same amount of water without the additive has evaporated under identical conditions by at least 2 hours.

5. A composition as claimed in claim 1, wherein the active ingredient which differs from the additive (A) is present in the composition in an amount in the range of from about 0.0001 to about 10% by weight, based on the total weight of the composition.

6. The composition as claimed in claim 1, wherein the crosslinked polymer (A1) of the additive is based on
(α1) about 0.1 to about 99.999% by weight, of polymerized, ethylenically unsaturated, acid-group-containing monomers or salts thereof or polymerized, ethylenically unsaturated monomers comprising a protonated or quaternized nitrogen,
(α2) 0 to about 70% by weight, of polymerized, ethylenically unsaturated monomers which are copolymerizable with (α1), and
(α3) about 0.001 to about 10, of one or more crosslinkers, the total of the amounts (α1) to (α3) being 100% by weight.

7. A process for the preparation of a composition as claimed in claim 1, wherein water is mixed with the additive in the form of a water-in-oil polymer dispersion (PD), which comprises a hydrophobic organic phase and crosslinked polymers distributed therein and is based on
(PD1) a crosslinked polymer in an amount in a range of about 10 to about 70% by weight, based on the total weight of the additive,
(PD2) a hydrophobic organic fluid in an amount in a range of from about 20 to about 80% by weight, based on the total weight of the additive,
(PD3) one or more water-in-oil emulsifiers in an amount in a range of from about 0.5 to about 10% by weight, based on the total weight of the additive,
(PD4) one or more oil-in-water emulsifiers in an amount in a range of from about 0.5 to about 10% by weight, based on the total weight of the additive,
(PD5) one or more auxiliaries in an amount in a range of from 0 to about 20% by weight, based on the total weight of the additive, and
(PD6) water in an amount of at least about 0.1% by weight, based on the total weight of the additive, but at least in such an amount that the total of (PD 1) to (PD6) amounts to 100% by weight,
and with at least one active ingredient which differs from the additive.

8. A composition which can be obtained by a process as claimed in claim 7

9. A method for increasing the efficacy of an active agent in a composition for controlling the growth of an organism which comprises adding from about 0.001 to about 2% by weight, based upon the total weight of the composition, an additive (A)
(A1) a crosslinked polymer in an amount in a range of from about 10 to about 70% by weight, based on the total weight of the additive (A),
(A2) a hydrophobic organic fluid in an amount in the range of from about 20 to about 80% by weight, based on the total weight of the additive (A),
(A3) one or more water-in-oil emulsifiers in an amount in a range of from about 0.5 to about 10% by weight, based on the total weight of the additive (A),
(A4) one or more oil-in-water emulsifiers in an amount in a range of from about 0.5 to about 10% by weight, based on the total weight of the additive (A),
(A5) one or more auxiliaries in an amount in a range of from 0 to about 20% by weight, based on the total weight of the additive (A), and
(A6) water in such an amount that the total of (A1) to (A6) amounts to 100% by weight.

10. A method for increasing the efficacy of a plant growth regulating agent in a composition for regulating the growth of plants which comprises adding from about 0.001 to about 2% by weight, based upon the total weight of the composition an additive (A)
(A1) a crosslinked polymer in an amount in a range of from about 10 to about 70% by weight, based on the total weight of the additive (A),
(A2) a hydrophobic organic fluid in an amount in the range of from about 20 to about 80% by weight, based on the total weight of the additive (A),
(A3) one or more water-in-oil emulsifiers in an amount in a range of from about 0.5 to about 10% by weight, based on the total weight of the additive (A),
(A4) one or more oil-in-water emulsifiers in an amount in a range of from about 0.5 to about 10% by weight, based on the total weight of the additive (A),
(A5) one or more auxiliaries in an amount in a range of from 0 to about 20% by weight, based on the total weight of the additive (A), and
(A6) water in such an amount that the total of (A1) to (A6) amounts to 100% by weight.

11. A method for synergistically increasing the efficacy of a composition for controlling the growth of organisms said composition comprising at least two active ingredients which comprising adding from about 0.001 to about 2% by weight, based upon the total weight of a composition, an additive (A)
(A1) a crosslinked polymer in an amount in a range of from about 10 to about 70% by weight, based on the total weight of the additive (A),
(A2) a hydrophobic organic fluid in an amount in the range of from about 20 to about 80% by weight, based on the total weight of the additive (A),
(A3) one or more water-in-oil emulsifiers in an amount in a range of from about 0.5 to about 10% by weight, based on the total weight of the additive (A),
(A4) one or more oil-in-water emulsifiers in an amount in a range of from about 0.5 to about 10% by weight, based on the total weight of the additive (A),
(A5) one or more auxiliaries in an amount in a range of from 0 to about 20% by weight, based on the total weight of the additive (A), and
(A6) water in such an amount that the total of (A1) to (A6) amounts to 100% by weight.

12. A method for synergistically increasing the efficacy of a composition for regulating the growth of plants said composition comprising at least two different plant growth regulators which comprises adding from about 0.001 to about 2% by weight, based upon the total weight of a composition, an additive (A)
(A1) a crosslinked polymer in an amount in a range of from about 10 to about 70% by weight, based on the total weight of the additive (A),
(A2) a hydrophobic organic fluid in an amount in the range of from about 20 to about 80% by weight, based on the total weight of the additive (A),
(A3) one or more water-in-oil emulsifiers in an amount in a range of from about 0.5 to about 10% by weight, based on the total weight of the additive (A),
(A4) one or more oil-in-water emulsifiers in an amount in a range of from about 0.5 to about 10% by weight, based on the total weight of the additive (A),
(A5) one or more auxiliaries in an amount in a range of from 0 to about 20% by weight, based on the total weight of the additive (A), and
(A6) water in such an amount that the total of (A1) to (A6) amounts to 100% by weight.

13. The composition according to claim 1, wherein the at least one ingredient which differs from the additive (A) is a herbicide, fungicide, bacteriacide, nematicide, insecticide, or viruscide.

14. A method for controlling or combatting the growth of an unwanted organsim which comprises applying to or bring into contact with said organism or an environment where it resides an effective amount of composition according to claim 13.

15. The method according to claim 14, wherein the composition is applied or brought into contact with the unwanted organism or to an environment where it resides by spraying.

16. The method according to claim 14, wherein the environment is a plant, a plant part, seed soil, or a smooth or rough surface.

17. The method according to claim 14, wherein the plants are fruit trees.

18. The composition according to claim 1, wherein the at least one active ingredient which differs from the additive (A) is a plant growth regulator.

19. A method for regulating the growth of a plant which comprises applying to or bringing into contact with said plant or to an environment where said plant resides an effective amount of the composition according to claim 18.

20. A method for controlling or combating the growth of unwanted organisms during storage or processing of fruit, crops or plant materials which comprises applying to said fruit, crops or plant materials or to an area where said fruit, crops, or plant materials are being stored or processed an effective amount of a composition according to claim 13.

21. A method for controlling the growth of unwanted organisms during the breeding of plants which comprises applying to said plants or to an area where said plants are being breed an effective amount of a composition according to claim 13.
